(19) United States
(12) Patent Application Publication
(10) Pub. No.: US 2007/0031463 A1
(43) Pub. Date: Feb. 8, 2007

(54) Methods and devices for releasing volatile substances

(76) Inventors: Spiros Fotinos, Athens (GR); Ligia Panaitescu, Attiki (GR); Ekaterini Tsardaka, Athens (GR); David Paul O'Halloran, Milltown, NJ (US)

Correspondence Address: BROMBERG & SUNSTEIN LLP, 125 SUMMER STREET, BOSTON, MA, 02110-1618, US

(21) Appl. No.: 10/547,068

(22) PCT Filed: Feb. 26, 2004
(86) PCT No.: PCT/US04/05602

§ 371 Date: Sep. 26, 2006

Related U.S. Application Data
(60) Provisional application No. 60/450,036, filed on Feb. 26, 2003.

(51) Int. Cl. A61K 9/28; A01N 25/00; A61K 9/70

(52) U.S. Cl. 424/405; 424/443; 427/2.14

(57) ABSTRACT

Embodiments of the invention are directed toward devices for emitting a volatile substance, and associated methods of producing such devices. In one embodiment, the delivery device for a volatile substance includes an active layer that is created from a dried polymer solution that contains the volatile substance. A release liner is in direct contact with the active layer, and covers a side of the active layer. The polymer solution may include thickeners, plasticizers, dyes, and preservatives. The devices may deliver volatile substances such as fragrances, masking odors, or therapeutic agents. Some embodiments of the invention are directed toward a patch by positioning a barrier layer on the active layer; the barrier layer may adhere to a surface, and isolate the surface from the active layer. Other embodiments are directed toward controlling the rate of delivery of the volatile agents from patches and other volatile delivery devices.

### TECHNICAL FIELD

The present invention relates to compositions and methods for the release of volatile substances to the environment and applications thereof.

### BACKGROUND ART

People have long applied scents and fragrances to themselves and their garments for a variety of reasons, e.g. to mask or enhance the natural odor of the user, to bring about a feeling of well-being, enhance people's mood, sedate or relax, repel insects, etc. Volatile substances can serve as simple fragrances for people to wear or for the overall refreshing of an environment. Volatile substances may also have therapeutic properties, offering medicinal or therapeutic effects such as relief from exemplary conditions including headache, sinus headache, small muscle tension, puffy oedematous eyelids, cold, nasal congestion, coughing, bronchitis, and asthma.

Notwithstanding, when deposited on the skin, mixtures of perfumes or fragrance raw materials lose their intensity and may change their character with time, mainly due to factors such as differential evaporation and skin penetration.

It is often desired to have a perfume-emitting article suitable for wearing on any part of a user's clothing or even skin, suitable for inclusion in a magazine, in a pouch, or on a postcard, as well as to provide a fragrance sample for marketing purposes.

With any of these uses, it would also generally be desirable for a fragrance-emitting article to exhibit the ability to release a fragrance at a suitable intensity for an extended period of time.

Additionally, it would often be desirable for the fragrance-emitting article to exhibit the ability to be reversibly adhered to an article of clothing during the time the fragrance is being released.

Volatile substances with beneficial properties to humans have had a history of popular use. At present, volatile substances are generally delivered in the form of an oil (bath or massage), candle wax or spray using a vaporizer or a diffuser. These methods are time consuming or cumbersome, as suggested by reduced compliance of the subjects to follow the mode of application.

Additionally, patches have been described for placing on a subject and releasing agents into the environment to act as deterrents for sharks (U.S. Pat. No. 5,616,333) and as insect repellants (U.S. Pat. No. 5,656,282).

An insect repellant containing device uses differential adhesion to separate a layer containing an absorbent material saturated with insect repellant fluid from a pouch on one surface, while maintaining contact with an adhesive backing layer on the opposite surface. A device or patch, containing insect repellant would be useful for providing high concentrations of the volatile agent into the environment without producing an adverse reaction on the skin of the subject. For example, in excess of 35%, N-diethyl-meta-toluamide can cause rashes. Heavy use of concentrations in excess of 80% have been linked to short term schizophrenia, while behavior will return to normal after use is discontinued. Many people have noticed becoming irritable after extended use of high concentrations of DEET based repellants.

A perfume patch for emitting a fragrance has also been formerly described (U.S. Pat. No. 4,880,690). Such a patch combines a fragrance and monomers that are polymerized into a fragrance-emitting layer composed of polyurethane and fragrance. The fragrance emitting-layer is difficult to tailor to specific requirements (e.g., requiring a specific rate of volatile emission, formulating specific volatile formulations depending upon needs) since a new polymerization is required to make each fragrance-emitting layer. Also, manufacturing of such a patch involves in situ polymerization and the concomitant difficulty of managing such a process (e.g., controlling the presence of catalysts and photoinitiators, curing the polymer, etc.).

A device for releasing vapors and scents is composed of a multilayer laminate that includes a reservoir layer, which may be gelled polymer, that incorporates the volatile substance (U.S. Pat. No. 5,071,704). Such a device requires the use of a diffusion rate limiting membrane to cover the reservoir layer to control release of a volatile substance into the environment. Thus different rate limiting membranes must be designed and manufactured to control the rate of volatile release.

It would be desirable to provide a patch for releasing volatile substances into the environment, which would be easy to use, protecting the user from skin contact with the volatile substance, and further, to offer flexibility to manipulate and tailor the release of the volatile agent over pre-selected periods of time. As well, methods which ease the effort of manufacturing an active layer, and ease the tailoring of the properties of an active layer to obtain a particular release rate, or other properties, would also be advantageous.

### SUMMARY OF THE INVENTION

In an embodiment of the invention, a method for making an active layer to deliver a volatile substance is presented. The method includes the steps of providing a polymer solution that has polymer, solvent, and a volatile substance; applying the polymer solution to a release liner; and drying the applied polymer solution to form the active layer. The active layer may be a gel, a semi-solid, or a solid. A barrier layer may be placed on an opposite side of the active layer from the release liner, optionally attached to the active layer with adhesive or through an adhesive property of the surface of the barrier layer.

The aforementioned method may also be used to make a patch. When the barrier layer is used, a side of the barrier layer opposite the active layer may have an adhesive layer that is covered by another release liner; the application of the adhesive and release liner may be completed before the barrier layer is attached to the active layer. In a particular embodiment, double coated tape may be used as the barrier layer.

In another embodiment of the invention, a method for making a patch with a fraction adhesion layer is described. This method includes the step of applying a fractional adhesion layer to the barrier layer on the opposite side from the active layer; the fractional adhesion layer may be formed before the barrier layer is attached to the active layer. The fractional adhesion layer has an adhesion area that is a fraction of the total area of a side of the barrier layer. A fractional adhesion layer may be created by applying adhesive to a fraction of the total area of a side of the barrier layer only. Alternatively a mask layer may be adhered to the barrier layer, and adhesive is applied to the mask layer to cover a fraction of the total area of a side of the barrier layer only. In another alternative, an adhesive layer is applied to the barrier layer. Subsequently, an intermittent mask layer is applied to the adhesive layer such that adhesive is left exposed on the adhesion area of the barrier layer. The intermittent mask layer may be a perforated film, a film with removed segments, or an intermittent protective coating. These embodiments may be practiced using double coated tape as the barrier layer. As well, these embodiments may be practiced in conjunction with the other embodiments of the invention describing active layers produced from polymer solutions.

More embodiments of the invention are directed to methods that describe the polymer solution used to form the active layer. The polymer may be hydrophilic, or may use water, alcohol, or glycol as a solvent. The polymer of the polymer solution may be a polyurethane, (e.g., polyurethane-1). The volatile substance may include one or more of an aromatherapy agent; an aromatherapy oil, a therapeutic agent, a deodorizer, a perfume, an insect repellant, a botanical extract, a botanical oil, and a masking odor.

Other components that may be used in the polymer solution, alone or in combination, include thickeners and plasticizers. Thickeners that may be used include xanthan gum, cellulose, polyvinyl pyrolidone, or carbomer. Thickeners may also be a combination of ammonium polyacrylate, isohexadecane and polyethylene glycol-40 castor oil; polyacrylamide, polydecene and ethoxylated lauryl alcohol; polyacrylamide, C₁₃₋₁₄ isoparafin and ethoxylated lauryl alcohol; or polyquaternium 32 and mineral oil. A glycol may be used as a plasticizer.

Another component that may be used in a polymer solution a rate controlling composition. In an embodiment of the invention where the polymer solution includes a rate controlling composition, drying the applied polymer solution results in the formation of a rate controlling active layer. Rate controlling compositions may include one or more of the following components: polymers, wax, silica, kaolin, chalk, diatomaceous earth, bentonite, titanium dioxide, glass particulates, and metal particulates. Polymers that may be used in the rate controlling composition include gum, polyolefin, polyvinyl pyrrolidone, ethylenevinyl acetate copolymer, polyether esteramide, cellulose derivatives, polyethylene, polyester, polystyrene, and polyamide. A rate controlling composition may also include an encapsulating device containing the volatile substance.

In another embodiment of the invention, a method for making an active layer to deliver a volatile substance in a controlled manner is described. The method includes the steps of providing a polymer solution that has a polymer, a solvent, and the volatile substance; applying the polymer solution to a side of a breathable layer, an opposite side of the breathable layer from the polymer solution being covered at least in part by a release liner, the breathable layer controlling the rate of delivery of the volatile substance; and drying the polymer solution to create the active layer.

Embodiments of the invention also include a method for making an active layer to deliver a volatile substance at an enhanced rate. In such an embodiment a polymer solution is applied to a release liner to create a surface with a high specific surface area. In particular, the polymer solution may be entrained with bubbles to create an active layer with a high specific surface area.

Various other embodiments of the invention relate to devices that deliver volatile substances, and patches, that correspond to products that may be produced by the methods described earlier.

In another embodiment of the invention, a patch with multiple release liners for delivering a volatile substance at a controlled rate is presented. The patch includes an active layer having the volatile substance, which may be a dried polymer solution; at least two release liner segments covering one side of the active layer; and a barrier layer disposed on an opposite side of the active layer from the at least two release liner segments. The release of the volatile substance depends upon the number of release liner segments removed from the active layer. The release liner segments may overlap each other, or be adjacent to each other. A breathable layer may be positioned between the release liner segments and the active layer to further hinder the rate of delivery of the volatile substance.

### BRIEF DESCRIPTION OF THE DRAWINGS

The foregoing features of the invention will be more readily understood by reference to the following detailed description, taken with reference to the accompanying drawings, in which:

FIG. 1 is depicts a cross-sectional view of a patch in accordance with an embodiment of the invention;

FIGS. 2A-2E depict views of various fractional adhesion layers, in accord with embodiments of the invention;

FIG. 3 depicts an embodiment of the invention that utilizes a porous or breathable layer in a patch;

FIGS. 4A-4F depict embodiments of the invention that utilizes a rectangular-shaped segmented release liner; and

FIGS. 5A-5D depict a concentric circle segmented release liners in accord with embodiments of the invention.

### DETAILED DESCRIPTION OF EMBODIMENTS

Embodiments of the patch described herein permit a volatile substance to be delivered without contacting the skin of a subject. An active layer is formulated from a polymer solution that includes a polymer in a solvent with a volatile substance. A combination of other components, such as thickeners, plasticizers, rate controlling compositions, and other components, may be formulated into the polymer solution. The polymer solution is dried to form an active layer that acts as a carrier for the volatile substance. The use of a barrier layer offers protection of the skin from the volatile substance contained in the active layer. This avoids irritation and sensitization of the skin, which may occur when a subject's skin is contacted with the volatile substance in liquid or solid form.

The patch provides controlled release of the volatile substance for desired time periods, for example, from as little as about one minute, or from about five minutes to one hour, or from one hour to four hours, or from four hours to twelve hours and from 12 hours to 24 hours and in another embodiment of the invention for at least 6 hours including embodiments desired to last for 12 hours or about 24 hours.

The vapors of the volatile substance may serve to bring about from a simple feeling of pleasure and well being, mood enhancement, sedation and relaxation, to a, feeling of actual relief from a condition including headache, sinus headache, small muscle tension, puffy oederiatous eyelids, cold, nasal congestion, coughing, bronchitis, asthma, and others. The vapors of the volatile substance may act so as to neutralize body odor, refresh the environment, aromatize a room space and the like, repel insects or parasites, etc.

As used in this description and the accompanying claims, the following terms shall have the meanings indicated, unless the context otherwise requires:

“Patch” refers to any of a multi-layered optionally laminated device for the delivery of agents including volatile substances.

“Natural” refers to anything derived from nature.

“Selected site” refers to any part of the body of a subject or any part of a location or object.

“Subject” refers to an animal, for example, a mammal or a human being.

“Agent” or “substance” refers to one or more parts of, or an entire, composition. The agent may be a single natural or synthetic chemical compound or a mixture of compounds with volatile properties.

“Volatile” refers to a tendency to enter a vapor phase.

The features and embodiments described herein refer to methods and devices for delivering a volatile substance from a patch. Such features and embodiments are intended to be merely exemplary and a number of variations and modifications will be apparent to those skilled in the art. All such variations and modifications are intended to be within the scope of the present invention.

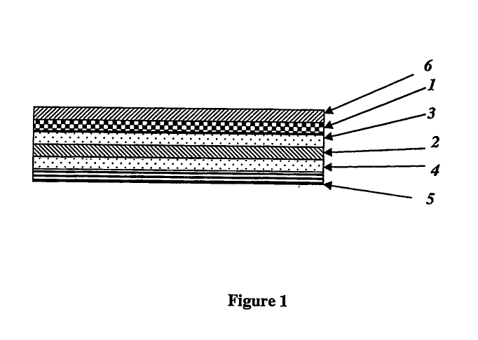

Some features of the invention will be more readily understood by reference to FIG. 1, which shows an embodiment of a patch for release of volatile substances. A patch is provided, that includes an effective amount of a volatile substance in an active layer 1, the active layer 1 being adhered by means of an adhesive layer 3 on a barrier layer 2, on the opposite side of the barrier layer 2, another adhesive layer 4 contacts a release liner 5. Another release liner 6 may be placed in contact with the active layer 1, opposite the adhesive layer 3, to prevent premature loss of the volatile substance. In other embodiments, the barrier layer 2 has adhesive properties so as to directly contact the release liner 5 such that an adhesive layer 3 is not utilized. In all of these embodiments, removal of the release liner 5 exposes an adhesive surface for adhesive contact with a surface of a subject or an object.

The volatile substance is included within the active layer. The volatile substance may have been mixed directly into the composition that formed the active layer or provided in embedded particles, micro-particles, microcapsules, nano-tubes, or other encapsulants embedded within the layer. With regard to the encapsulants, the volatile substance may be contained within the encapsulants' core, dispersed throughout their mass or positioned on their surface.

Volatile substances used in compositions for forming the active layer may include, but are not limited to, aromatherapy agents, aromatherapy oils, therapeutic agents, deodorizers, perfumes, and insect repellants. Sources for volatile substances include, but are by no means limited to, botanical extracts, botanical oils, and their derivatives.

Any aromatherapy agent known, in the art may be used with the embodiments of the invention. Examples include Lavendar, Camomille, Clary Sage, Frankincense, Marjoram, Melissa, Neroli, Rose and Ylang, Ylang, Bergomot, Cajeput, Garlic, Manuka, Niaouli, Ravensara, Tea-tree, Geranium, Grapefruit, Jasmine, Mandarin, Mimosa, Orange, Petilgrain, Sandalwood, Myrrh, Juniper, Cardomon, Vetivert, Cedarwood, Cypress, Thyme, Peppermint, Rosemary, Eucalyptus, Litsea cubeba, Fennel, Birch, Hyssop, Benzoin, Lemon, Pimento, and Ginger. Volatile compositions for use in a patch for acting as insect repellants include citronella, basil essential oil, geranium essential oil, lavender, rhodinol, geraniol, citronellol, citral, benzil, benzylbenzoate, N-butylacetanilide, Idalone, (FMC Corp, USA) N-iethyl-meta-toluamide (DEET), dimethyl carbate derivatives of cyclohexanol, 2-ethyl-2-butyl-1,3-propandiol, 2,5-dimethyl-2,5-hexandiol and similar higher alcohols, butyl esters or bicarboxylic acid of the formula H₉C₄—O—OC—(CH₂)ₙ—CO—O—C₄H₉ where n is an integer between 2 and 6 and mixtures thereof, and dimethyl pthalate or 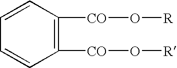
where R and R′ are C₁-C₅ alkyles.

Fragrances may also be included in the compositions for forming the active layer to mask unpleasant odors associated with the composition, or to increase the to attractiveness of a product in conjunction with marketing the product. Alternatively, fragrances may constitute the volatile substance itself; in such an instance, embodiments of the invention may be a fragrance patch for delivering a fragrance without contacting the fragrance on the skin of a person. Examples of sources of fragrances include essential oils, perfumes, and commercially available fragrances. Specific fragrances include, but are not limited to, peppermint, lime, and other citrus fruits. Preferably, the composition may contain 0 to 40 weight percent fragrance after drying.

In accordance with embodiments of the invention, the compositions for forming the active layer contain an optimum concentration of a polymer. Polyurethanes are a group of polymers that may be utilized. Polyurethanes are copolymers of isophthalic acid, adipic acid, hexanediol glycol, neopentyl glycol, amines, etc, and are known for their capacity to form films.

If needed; thickeners are added to the compositions to modify the viscosity of the composition and optimize its mechanical properties. Thickeners suitable for use in the compositions include, but are by no means limited to, any polymer or thickener conventionally used in cosmetics. Preferably, the thickeners may include xanthan gum; cellulose; polyvinyl pyrolidone; carbomer; mixtures such as (1) ammonium polyacrylate, isohexadecane and polyethylene glycol-40 castor oil (an example of such a mixture is available from Seppic under the trademark Simulgel A); (2) polyacrylamide, polydecene and ethoxylated lauryl alcohol (an example of such a mixture is available from C.I.T. Sarl under the trademark Ceragel EZ-7); (3) polyacrylamide, C₁₃₋₁₄ isoparafin and ethoxylated lauryl alcohol (an example of such a mixture is available from Seppic under the trademark Sepigel 305); and (4) polyquaternium 32 and mineral oil (an example of such a mixture is available from Ciba under the trademark Salcare SC-92). Preferably, the compositions may contain 0 to 10 wt % thickeners after drying.

Solvents are added to the compositions that eventually form the active layer, creating a polymer solution. Solvents suitable for use in the compositions include, but are by no means limited to, water, an alcohol (e.g., ethanol, isopropanol), glycol and mixtures thereof. The polymer solution may be hydrophilic. Preferably, the compositions include water and ethanol as the solvent. The solvent fraction of the solution is approximately 40 to 85 wt %, preferably between 50 and 75 wt %.

The compositions of the active layer may contain plasticizers, thickeners, dyes, pigments, surfactants, emulsifiers, fragrances, chelating agents, UV absorbers, antioxidants, preservatives, and mixtures thereof.

Plasticizers may be added to the compositions to modify the properties and wear time of the resultant compositions. Plasticizers suitable for use in the compositions include, but are by no means limited to, any water-binding ingredients generally used in cosmetics. Preferably, the plasticizers may include glycols, for example glycerin, propylene glycol and 1,3-butylene glycol; sorbitol, sodium polycarboxcylic acid, and sodium citrate. Most preferably, the plasticizer may include polypropylene glycol-10 methyl glucose ether. Polypropylene glycol-10 methyl glucose ether suitable for use in the compositions is available from Amercol under the trademark Glucam P-10. Preferably, the compositions may contain 0.5 to 20 wt % plasticizers after drying.

Dyes/pigments suitable for use in the compositions include, but are by no means limited to, any inorganic dyes, organic dyes, pigments and opacifiers conventionally used in cosmetics. Preferably, the dyes/pigments may include titanium dioxide, zinc oxide, iron oxide, D&C and FD&C dyes. Preferably, the compositions may contain 0.01 to 10 wt % dyes/pigments after drying.

Surfactants/emulsifiers suitable for use in the compositions include, but are by no means limited to, any anionic, cationic and nonionic surfactants and emulsifiers conventionally used in cosmetics. Preferably, the surfactants/emulsifiers may include ethoxylated alcohols, sodium lauryl sulfate and polyquaternium-31. Preferably, the compositions may contain 0.5 to 10 wt % surfactants/emulsifiers after drying. Surfactants/emulsifiers may be added to the compositions to stabilize the composition.

Chelating agents suitable for use in the compositions include any chelating agents conventionally used in cosmetics. Preferably, the chelating agents may include ethylenediaminetetraacetic acid, ethylenediaminetetraacetic acid-disodium salt and ethylenediaminetetraacetic acid-tetrasodium salt. Preferably, the compositions may contain 0.01 to 1 wt % chelating agents after drying. Chelating agents may be added to the compositions to bind metal ions and to promote the overall stability of the compositions.

UV absorbers suitable for use in the compositions include any water and/or oil soluble sunscreen conventionally used in cosmetics. Preferably, the UV absorbers may include benzophenone-3 and benzophenone-4. Preferably, the compositions may contain 0.1 to 4 wt % UV absorbers after drying. UV absorbers may be added to the compositions to protect them from any decomposition and/or change in their organoleptic properties, such as color, etc., that may be caused by the deleterious effects of exposure to ultra-violet light.

Antioxidants suitable for use in the compositions include any antioxidants conventionally used in cosmetics. Preferably, the antioxidants may include tocopherol, tocopherol acetate, propyl gallate, butylated hydroxyanisole and butylated hydroxytoluene. Preferably, the compositions may contain 0.05 to 3 wt % antioxidants after drying. Antioxidants may be added to the compositions to combat the deleterious effects of oxidation.

Preservatives suitable for use in the compositions include any preservative or preservative system conventionally used in cosmetics. Preferably, preservatives suitable for use in the compositions may include a mixture of phenoxyethanol, methylparaben, isopropylparaben, isobutyparaben and butylparaben (an example of such a mixture suitable for use is available from, ISP/Sutton under the trademark LiquaPar Optima). Preferably, the compositions may contain 0.2 to 3 wt % preservatives after drying. Preservatives may be added to the compositions to combat microbiological contamination.

In order to form the active layer, the composition is dried. The drying process may occur at room temperature or in an oven. The compositions, after drying, preferably contain 1 to 40 wt % volatile substances, 5 to 90 wt % polyurethane and 0 to 10 wt % thickeners.

The active layer holds the volatile substance in place, unlike patches in which the volatile substance may be in microcapsules or pellets free to move about within a chamber. In accordance with a variety of embodiments, the active layer may be a gel; a semi-solid that is not a gel; a solid that may be a film or a disk, whereby the film or the disk may be thin or thick, flexible or rigid; or any other material that has a tendency to retain a particular configuration after being subjected to an external force.

In the embodiment provided in FIG. 1, adjacent to the active layer 1 and sandwiched between two adhesive layers 3, 4 is a barrier layer 2. The barrier layer 2 may be any substrate that cannot be penetrated by the volatile compound. Possible materials for use as a barrier layer 2 include plastic, coated fabric, paper, cellophane, polyethylene, polyester, polypropylene, polyurethane, polyvinyl chloride, polyamide or metallic foils such as aluminum foil. Examples of a barrier layer 2 include, but are not limited to, a single film, a combination of films, and a film of tape double side coated with adhesive. In a particular embodiment of a barrier layer 2, the thickness of layer 2 is 1 to 5 mils ( 1/1000 of an inch is 1 mil).

One or both of the adhesive polymeric layers 3, 4 can be made of synthetic adhesives such as acrylics, rubber, silicone, or other suitable materials that may have pressure sensitive properties, and which are inert materials, biologically and topically acceptable. Preferably, topically acceptable polymers with adhesion properties include acrylic based polymers such as GELVA series sold by Solutia, rubber based polymers such as DURO-TAK series sold by National Starch and silicone-based polymers such as BIO-PSA X74302 SILICONE PSA sold by Dow Corning.

Similarly, one or both of the adhesive polymeric layers 3, 4 may be formed from entirely natural materials, a plant protein such as prolamine (gliadin from wheat or zein from corn), and plant polar lipids such as ceramides. They may also be formed by other polysaccharides including cellulose and cellulose derivatives; cyclodextran, gums such as arabic gum, tragacanth gum, chatti gum, karaya gum mastic gum; or gums produced by a microbial growth and fermentation such as xanthan gum, gellan gum. An entirely natural sheet, such as cellulose can also be used.

The adhesive polymers may consist of at least one layer of the adhesive, containing substances and/or additives. The adhesive polymer may be composed of more than one layer with a dry thickness in the range of 0.5-3.0 mils or, more particularly, 0.5-2.0 mils. An adhesive layer 3, 4 may be a continuous film or a discontinuous film in the form of a random or non-random pattern.

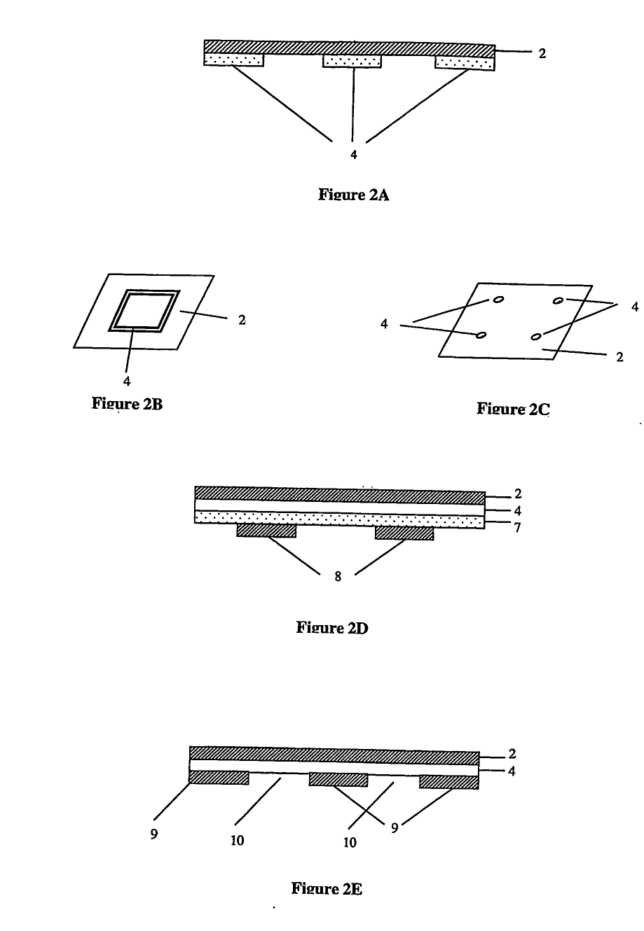

Some embodiments of the invention limit the area of contact between an adhesive layer and an external surface (e.g., skin of a person) through the use of a fractional adhesion layer that creates an adhesion area covering only a fraction of the surface of a barrier layer. A release liner may be applied to cover the adhesion area, the liner being removed prior to use. Such embodiments may minimize the risk of adverse effects (e.g., mechanical or physiological) that the adhesive may have on the surface, while allowing a patch to adhere to the surface. In one embodiment of the invention shown In FIG. 2A, the adhesive layer 4 in contact with a barrier layer 2 is applied such that only a selected fraction of the barrier layer surface is covered by the adhesive. The pattern of the adhesive layer may be continuous (as shown in FIG. 2B) or discontinuous (e.g., a pattern of unconnected dots or lines as shown in FIG. 2C). The pattern of the adhesive layer may be chosen to assure a desired amount of adhesion between a patch and an external surface, while minimizing the amount of adhesive used. Alternatively, the embodiment of the invention may utilize a mask layer 7 that adheres to an adhesive layer 4, as shown in FIG. 2D. The mask layer prevents or limits the adhesion of an adhesive layer where the mask layer covers the adhesive layer. In one example, the mask layer 7 entirely covers the adhesive layer 4. The opposite side of the mask layer from the adhesive layer 4 is treated with an adhesive 8 that only covers a selected portion of the mask layer surface, in accord with what is described above for the barrier layer. These embodiments may be implemented by using a printing press with an embossed roller to apply the specific pattern of adhesive to the barrier layer or the mask layer. As well, these embodiments may be implemented by applying the pattern of adhesive before the barrier layer is attached to an active layer, or after the barrier layer is attached.

Other embodiments of the invention may utilize an intermittent mask layer. As shown in FIG. 2E, an intermittent mask layer 9 covers the adhesive layer 4. The intermittent mask layer 9 is configured with one or more openings 10 such that the adhesive layer is exposed at every opening 10 when the intermittent mask layer does not cover the adhesive layer 4. The intermittent mask layer 9 may be composed of one continuous film or a set of discrete films arranged on the adhesive layer 4. Thus, only a portion of the adhesive layer 4 is exposed to contact with an external surface when a patch is applied. In one example, an intermittent mask layer may utilize a film that is perforated, or a film in which segments of the film have been removed (e.g., cut out). This prepared film is applied over the adhesive layer, the perforated or open segmented areas exposing a fraction of the adhesive layer. Alternatively, a film may be applied to cover the entire adhesive layer, followed by a step of perforating, or removing segments of, the film to expose the fraction of the adhesive layer.

In a related embodiment, the intermittent mask layer may take the form of an intermittent protective coating that is printed onto the adhesive layer, preferably by a continuous process. For example, the intermittent protective coating may be SVL01002 by Akzo, a specially formulated, high gloss, solvent-based overprint, containing 11122 FDA approved varnish. The intermittent protective coating may be applied to the adhesive layer using a printing press. An embossed roller may be used to apply the protective coating to the adhesive layer. The a real coverage of the intermittent protective coating that covers the adhesive layer may be adjusted to control the adhesion of the patch. The pattern applied by the printing process may take on any shape or size suitable to obtain a desired adhesion of the patch. In a particular embodiment, a double-coated tape may serve as a barrier layer for a patch. One adhesive side of the double-sided tape is exposed. Varnish is applied to the exposed adhesive using a printing press (e.g., Flexographic printing plate and press) to decrease the attraction of the exposed adhesive layer to a contacted surface. The amount and pattern of varnish applied to the exposed adhesive surface may be selected to adjust the adhesion of the patch to a contacted surface. A new release liner may be applied over the varnished adhesive layer to create a new double coated tape. The new double coated tape may be rolled up and later utilized to make a patch, in accord with other embodiments of the invention described herein.

Referring back to FIG. 1, the adhesive layer 4 on the external face of the barrier layer 2 is covered by a release liner 5, removed prior to the application of the patch to expose the adhesive surface for adhering to the desired application site, a subject or an object. Another release liner 6 may cover the active layer 1, preventing premature release of the volatile substance to the environment. Instead of, or in addition to, release layer 6, patches may be individually, or in groups, sealed in pouches.

The release liners 5, 6 may be made from substrates such as plastic, coated fabric, paper, cellophane, polyurethane, polyvinyl chloride (PVC), polyvinylidene chloride (PVDC), polyamide or metallic foils such as aluminum foil, preferably from polystyrene film (e.g. natural high impact grade, code 10106, by REXAM Release) or a siliconized polyester film. In accordance with a specific embodiment, the release liner is a “polyester” plastic coated with silicon anti-adherent coating (PET). In another specific embodiment, the thickness of the release liner is 3 to 10 mils. Embodiments of release liners include a single continuous layer, a plurality of segments that are adjacent to one another, and a plurality of segments in which each segment overlaps at least one other segment to some degree.

In another embodiment of the invention, the patch can include an active layer in which the dried polymer containing the volatile substance is itself adhesive. In this circumstance, the adhesive active layer will be adjacent and in contact with the barrier layer without an additional adhesive layer. If the barrier layer is adhesive, then the barrier layer may be in direct contact with its release liner.

Referring to FIG. 1, in an embodiment of the invention, a method is provided for making a device to release a volatile substance. A polymer, mixed with a solvent and a volatile substance, is applied on a first release liner 6 and dried to form an active layer 1. The device may be made into a patch by laminating a barrier layer 2 on the active layer 1 opposite the release liner 6.

Applying the polymer solution directly to a release liner may accrue the advantage of facilitating the formation of an active layer. Applying the polymer solution to a barrier layer may require the application of an adhesive between the solution and barrier layer, which may increase the difficulty of forming a smooth homogeneous active layer. Such a smooth layer promotes ease of manufacturing in automated devices by minimizing resistance introduced by the layer and eliminating the possibility of solid agglomeration that may be present with the use of an adhesive. As well, a smooth surface is well characterized for understanding the transport of a volatile substance from a layer, as opposed to a varying inhomogeneous surface. If an adhesive is not used, formulations of the polymer solution are restricted to those having self-adhesive properties to insure bonding of the active layer and barrier layer.

In a particular embodiment of the method, a polymer is mixed with a solvent and a volatile substance to make an aqueous polymer solution. Other components may also be added to complete the composition for the active layer. The composition is applied to a substrate, either a release liner 6 or a barrier layer 2.

Particular method steps can include applying a pressure sensitive adhesive 3 to a side of the barrier layer 2. Given an active layer 1 on a release liner 6, the barrier layer 2 is disposed over the active layer 1. This involves laminating the side of the active layer 1 opposite the release liner 6 against the side of the barrier layer 2 having the pressure sensitive adhesive 3. Further particular method steps include applying a pressure sensitive adhesive 4 to another release liner 5. The side of the release liner 5 with the pressure sensitive adhesive 4 is laminated to the side of the barrier layer 2 opposite the active layer 1. The type of adhesive used in 3, 4 may be the same or different.

In another embodiment of the above invention, the barrier layer 2 and the release liner 5 adjacent to it may be laminated as a single product before the barrier layer 2 is laminated to the active layer 1.

In an embodiment of the invention, a method is provided for making a patch for the release of volatile substances, beginning with an aqueous polymer solution, that includes (a) mixing a volatile substance with the aqueous polymer solution, (b) addition of a thickener which leads to a significant viscosity build-up, (c) coating of the mixture obtained onto a release liner (e.g. polyester) and drying of the solution to create an active layer, d) in parallel, coating of a release liner (e.g. PET of 3 mil) with a pressure sensitive adhesive (e.g. Gelvao 737), followed by drying of the adhesive and lamination with the “barrier film” (e.g. PET of 1 mil); in the meantime, coating of another release liner (PET of 3 mil) with a pressure sensitive adhesive (e.g. Gelvao 737), followed by drying of the adhesive and lamination on the free side of the “barrier” film, e) delaminating one of the PET (3 mil) layers from the barrier layer so as to leave the adhesive facing the free surface of the active layer and f) lamination of the active layer with the adhesive side of the barrier layer system described at e).

In another embodiment of the invention, instead of putting together the system described at d), a commercial tape is used (e.g., polyethylene-based Tape 1512 by 3M or polyester-based Tape 1513 by 3M). The paper layer of the double-coated tape should be removed and laminated with the adhesive side facing the free surface of the active layer.

The embodiments describing methods for making a patch may also be performed in a continuous process that produces a plurality of patches simultaneously. In an instance where the method of making a plurality of patches in a continuous process produces the plurality of patches connected as layers of continuous sheets, a step of cutting the sheets to form a plurality of discrete segments, each segment being a patch, is included.

The patch may be formed as a comfortable, cosmetically elegant patch for use on the skin of a subject. The size and the shape of the patch containing volatile substance may be designed to fit the site of application. The size, shape, and color of the device can be fanciful, or can be manufactured for minimal contrast with a shade of the skin. In an embodiment of the invention, the thickness of the patch, as shown in FIG. 1, lies between 10 and 50 mils.

In further embodiments, the volatile substance may be released from the patch over a twenty-four hour period, a twelve-hour period, a six hour period or a one hour period. The time period for release of the volatile substance may be adjusted by altering one or more of the concentration of the volatile substance, the polymer, the thickener, or a rate controlling composition in the active layer. The thickness of the active layer, or the time in which the volatile substance is allowed to stabilize with other rate controlling components, may also affect the rate of evaporation of the volatile substance from the patch.

In particular embodiments, incorporation of the volatile substance in microcapsules, nanotubes, microsponges, microparticles, or other encapsulating devices serves to control the release of the volatile substance. In certain embodiments, the volatile substance is pre-ispersed in a rate controlling composition. Such a composition may minimize loss of the volatile material during drying, and may control the release of the volatile material during actual use by altering the effective release rate of the volatile material. One example of a rate controlling composition is a lipophilic material comprising, but not limited to, a polyethylene powder of suitable particle size, fumed silica, or silicon dioxide. In specific embodiments, the polyethylene powder may be a polyethylene homopolymer, such as type A6 by Honeywell; or a micronised polyolefin, such as types A6 and C30 by Acumist. This dispersion may be left for a sufficient time interval, ranging from a few hours to a few days, to stabilize, and may be subsequently mixed with the other components of the composition; the time of stabilization may be used to influence the release rate of a volatile composition in an active layer, or specific components of the volatile composition. Other polymers may also serve as rate controlling compositions; examples include gums, polyolefins, polyvinyl pyrrolidones, ethylenevinyl acetate copolymers, polyether esteramide, cellulose derivatives, polyethylenes, polyesters, polystyrenes, and polyamides. More examples of rate controlling compositions include waxes, silica, kaolin, chalk, diatomaceous earth, bentonite, titanium dioxide, glass particulates, and metal particulates. The amount of a rate controlling composition may be adjusted to control the desired rate of delivery, or profile of rate of delivery as a function of time, of the volatile substance. As well, a combination of components, as described above, may be used to achieve a desired controlled rate of volatile release. Use of particular rate controlling compositions may alleviate the need to use a thickener in certain instances.

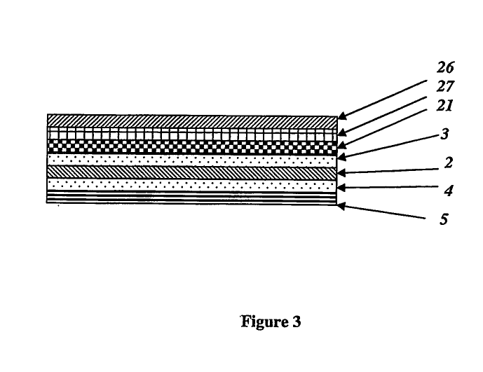

As is known in the art, control of the release rate of the volatile substance may also be achieved by the use of an adjacent porous or a breathable layer 27 placed on top of the active layer 21 on the side that releases the agent to the environment, as shown in FIG. 3. In such an embodiment, a polymer solution may be applied to the porous or breathable layer 27, the layer 27 acting as a substrate, and the solution dried to form the active layer 21. Layer 27 reduces the rate at which the volatile substance is allowed to evaporate; adjustment of the adjacent layer's type and thickness will affect the release rate of the volatile substance. A release liner 26 may be added over layer 27 in order to prevent premature release of the volatile substance. In these cases, an adhesive layer 28 may be needed between layer 27 and release liner 26.

Control of the amount of volatile substance released in the environment may also be achieved by providing a release liner formed by multiple segments. The amount of the volatile substance released depends upon the surface of the active layer uncovered from removal of one or more of the release liner segments. Thus, a user may choose the rate of release of the volatile substance by removing particular segments. In such embodiments, the release liner may be composed of at least two segments, preferably three, most preferably more than two. The segments may be of the same or different size, shape and color. The segments are positioned in a manner that facilitates serial, individual removal thereof. They may be relatively loosely connected or attached to each other, adjacent to each other, partially or fully overlapping each other, or placed on top of each other in a homocentric or an eccentric manner. The segments may also be configured in a way that allows removal thereof in groups, and their removal may be facilitated by the use of tabs.

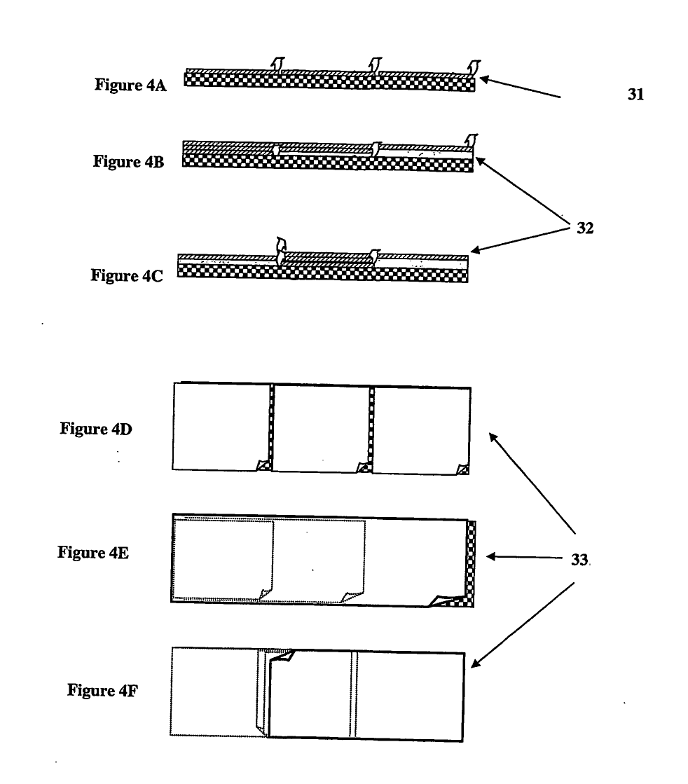

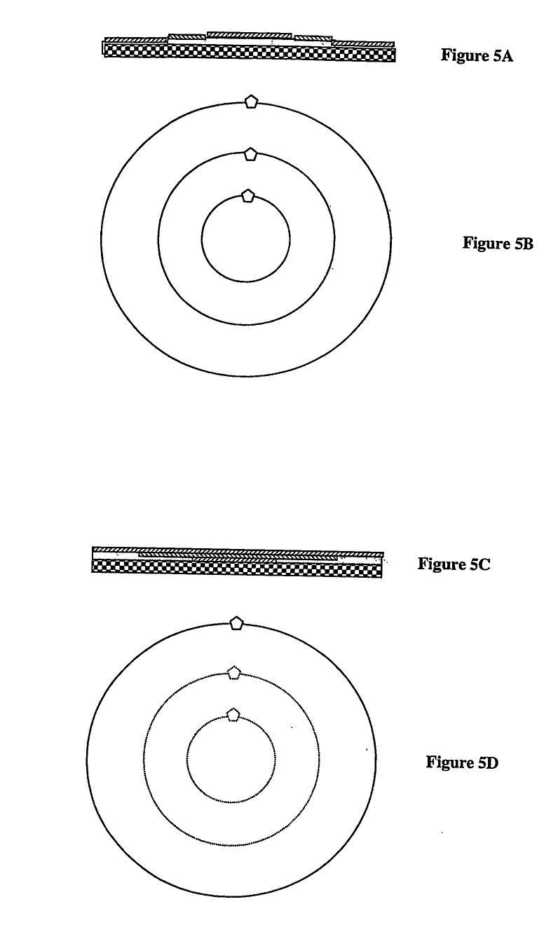

FIGS. 4A-4F provide illustrations of a controlled release device 33 utilizing a three-segment release liner. The segments may be overlapping 32 (a side and top view of one embodiment shown in FIGS. 4B and 4E respectively, and a side and top view of another embodiment shown in. FIGS. 4C and 4F respectively) or may be adjacent to one another 31 (side and top views shown in FIGS. 4A and 4D respectively). The removal of each segment exposes a portion of the active layer, which facilitates evaporation of the volatile component. FIGS. 5A-5D provide illustrations of another controlled release device utilizing a three-segment release liner in which the release liner consists of one central circular segment and two concentric ring segments. FIGS. 5A and 5B depict a side and top view of an embodiment where the segments are adjacently arranged. FIGS. 5C and 5D depict a side and top view of another embodiment where the segments overlap. Again, the removal of each segment exposes a portion of the active layer wherefrom the volatile component evaporates.

If additional control of the release of the volatile substance is needed, the embodiments utilizing release liners formed by multiple segments may also include a porous or breathable layer placed adjacent to the active layer on a side opposite the barrier layer, as mentioned above. Again, in these cases, an adhesive layer may be needed between the breathable layer and release liner.

Embodiments of the invention may also include patches that promote the transfer of a volatile substance into the atmosphere. Such an embodiment includes creating an active layer with high-specific surface area to increase the area for emission of the volatile substance relative to a flat or smooth surface. For example, a polymer solution may be agitated by intense stirring, or gas bubbling (e.g., nitrogen), to create a bubble entrained solution. The solution may be applied to the release liner and dried. The result is the formation of an active layer with an increased specific surface area due to the presence of pores, resulting from the bubbles, relative to the specific surface area of the dried solution if bubbles were not entrained. The size and number of bubbles per unit volume may be altered to control the rate of release of the volatile substance. Any technique known to those of ordinary skill in the art may also be applied to create a high-specific surface area on a surface of the active layer.

In further embodiments, the patch may be, individually or in groups, sealed in pouches. A patch should be removed from its pouch immediately prior to the use of the patch. Additional protection, of the volatile substance from being released prematurely to the environment is thus, offered, with or without the presence of the release liner on the active layer surface. The pouches may be formed from materials that are impermeable to gas or liquids and may have an easy opening to provide access to the patch. In an embodiment of the invention, pouches are made from aluminum foil, paper and polyethylene, preferably low density polyethylene, or polyethylene terephthalate or polyamide. The pouches are optionally coated with Barex resin. In other embodiments of the invention, pouches are made from polypropylene, metal-coated PET, or PET covered with one or more metal oxides.

The following examples are provided by way of illustrating embodiments of the invention but are not intended to be limiting.

### EXAMPLES

### Example 1

### A Patch Containing a Fragrance in an Active Layer

A patch containing a fragrance in an active layer was prepared as follows:

The fragrance was dissolved in a polyurethane solution (Luviset P.U.R., by BASF), containing 30% polyurethane-1, 10% ethanol and 60% water, under continuous stirring. Erythrosine was added as a colorant. A thickener, Sepigel 305 (by Seppic), was also added under stirring. A viscous solution was obtained. Using a coating device with a 10 mils casting gap, a layer of the viscous solution was coated on the siliconized side of a polyester film (PET of 3 mil). Following drying in an oven at 60° C. for 12 minutes, an active layer was formed on the polyester film, resulting to roll A.

A medical tape (1512, by 3M), consisting of a 1.5 mil transparent polyethylene film coated on both sides with a hypoallergenic pressure-sensitive acrylate adhesive, was laminated on the surface of the active layer. The side of the medical tape opposite the active layer was covered by a double side coated bleached Kraft-Glassine silicone paper liner. The paper liner is removed immediately prior to use, exposing an adhesive surface for application on the desired site. The product may be delaminated by removing the polyester film. Patches were subsequently cut to the desired dimensions. Alternatively, in the above example, the polyester film may be maintained on the active layer.

Detailed composition of the active layer of the above example is given below:

| Materials (solids) | % (on a dry basis) |
|---|---|
| Luviset P.U.R. | 69.08 |
| Fragrance H 8734 | 29.94 |
| Sepigel 305 | 0.94 |
| Erythrosine | 0.04 |
| Total | 100.00 |

### Example 2

### A Patch Containing a Fragrance in an Active Layer

A mixture was prepared following Example 1, except that less Sepigel 305 was added and a less viscous mixture was obtained. An active layer was prepared following Example 1, except that drying was performed for 17 minutes. A patch was also thereafter produced following Example 1.

Detailed composition of the active layer of the above example is given below:

| Materials (solids) | % (on a dry basis) |
|---|---|
| Luviset P.U.R. | 69.31 |
| Fragrance H 8734 | 30.05 |
| Sepigel 305 | 0.60 |
| Erythrosine | 0.04 |
| Total | 100.00 |

### Example 3

### A Patch Containing an Encapsulated Fragrance in an Active Layer

An encapsulated fragrance (Buttercream by LIPO, USA) was utilized in the active layer. The polyurethane solution and thickener of Example 1 were utilized. The active layer was prepared following Example 1, except that drying at 60° C. was performed for 20 minutes. The viscous mixture was coated on the non-siliconized side of a polyester film. A patch was thereafter produced following Example 1.

Detailed composition of the active layer of the above example is given below:

| Materials (solids) | % (on a dry basis) |
|---|---|
| Luviset P.U.R. | 71.25 |
| Encapsulated fragrance | 20.43 |
| Sepigel 305 | 8.32 |
| Total | 100.00 |

### Example 4

### A Patch Containing an Encapsulated Fragrance in an Active Layer

The same encapsulated fragrance as in Example 3 was used. A mixture was prepared as in Example 3, except that less Sepigel 305 was added and a less viscous mixture was obtained. An active layer was prepared as in Example 3, except that drying was performed for 25 minutes. A patch was thereafter produced as in Example 3.

Detailed composition of the active layer of the above example is given below:

| Materials (solids) | % (on a dry basis) |
|---|---|
| Luviset P.U.R. | 73.51 |
| Encapsulated fragrance | 21.24 |
| Sepigel 305 | 5.25 |
| Total | 100.00 |

### Example 5

### A Patch Containing a Fragrance Formulation in an Active Layer

A fragrance formulation (V 20103/S) supplied by VIORYL (Greece), was used. The polyurethane solution of Example 1 was used; a thickener was not added since it was not needed. An active layer was prepared following Example 1, except that drying at 60° C. was performed for 14 minutes. A patch was thereafter produced following Example 1.

Detailed composition of the active layer of the above example is given below:

| Materials (solids) | % (on a dry basis) |
|---|---|
| Luviset P.U.R. | 69.98 |
| Product V 20103/S | 30.02 |
| Total | 100.00 |

### Example 6

### A Patch Containing a Fragrance Formulation in an Active Layer

The same fragrance formulation was used as in Example 5 except that less fragrance was utilized. A mixture was prepared following Example 5. An active layer was prepared as in Example 5, except that drying at 60° C. was performed for 20 minutes. A patch was thereafter produced following Example 5.

Detailed composition of the active layer of the above example is given below:

| Materials (solids) | % (on a dry basis) |
|---|---|
| Luviset P.U.R. | 80.00 |
| Product V 20103/S | 20.00 |
| Total | 100.00 |

### Example 7

### A Composition Releasing a Fragrance in a Controlled Manner

A formulation was prepared by mixing a perfume with Aerosil 200. The formulation was allowed to stabilize for 4 hours, followed by the addition of polyurethane and the colorant under careful stirring. The composition was subsequently coated as in Example 1 and dried at 60° C. for 20 minutes. An opaque film was obtained. Detailed composition of the fragrance-containing film of this example is given below:

| Materials (solids) | % (on a dry basis) |
|---|---|
| Polyurethane | 64.11 |
| Fragrance | 27.78 |
| Sepigel 305 | 0.40 |
| Aerosil 200 | 7.68 |
| Erythrosine | 0.03 |
| Total | 100.00 |

### Example 8

### A Patch Releasing a Fragrance in a Controlled Manner

A formulation was prepared by mixing a perfume with polyolefin A-6 by Honeywell. The formulation was allowed to stabilize for 4 hours, followed by the addition of polyurethane and the colorant under careful stirring. The composition was subsequently coated as in Example 1 and dried at 60° C. for 20 minutes, resulting in roll A. A medical tape (1513, by 3M), consisting of a 1.0 mil clear polyester film coated on both sides with a hypoallergenic pressure-sensitive acrylate adhesive, was laminated on the surface of the active layer. The side of the medical tape opposite the active layer was covered by a double side coated bleached Kraft-Glassine silicone paper liner. Patches were subsequently cut to the desired dimensions. Detailed composition of the fragrance-containing film of this example is given below:

| Materials (solids) | % (on a dry basis) |
|---|---|
| Polyurethane-1 | 55.88 |
| Fragrance | 24.22 |
| Sepigel 305 | 0.31 |
| Polyolefin A-6 | 19.56 |
| Erythrosine | 0.03 |
| Total | 100.00 |

### Example 9

### A Patch Releasing a Fragrance in a Controlled Manner

A formulation was prepared by mixing a perfume with polyolefin A-6 by Honeywell. The formulation was left overnight to stabilize, followed by the addition of polyurethane-1 and the colorant under careful stirring. No thickener was needed. The composition was subsequently coated as in Example 1 and dried at 60° C. for 30 minutes, resulting in roll A. A medical tape (1513, by 3M), consisting of a 1.0 mil clear polyester film coated on both sides with a hypoallergenic pressure-sensitive acrylate adhesive, was laminated on the surface of the active layer. The side of the medical tape opposite the active layer was covered by a double side coated bleached Kraft-Glassine silicone paper liner. Patches were subsequently cut to the desired dimensions. Detailed composition of the fragrance-containing film of this example is given below:

| Materials (solids) | % (on a dry basis) |
|---|---|
| Polyurethane-1 | 46.86 |
| Fragrance | 20.31 |
| Polyolefin A-6 | 32.80 |
| Erythrosine | 0.03 |
| Total | 100.00 |

### Example 10

### A Patch Releasing a Fragrance to the Environment

A formulation was prepared by mixing a perfume with polyurethane-1 and a colorant. No thickener was added. The composition was subsequently coated as in Example 1 and dried at 60° C. for 15 minutes, resulting in roll A. A medical tape (1513, by 3M), consisting of a 1.0 mil clear polyester film coated on both sides with a hypoallergenic pressure-sensitive acrylate adhesive, was laminated on the surface of the active layer. The side of the medical tape opposite the active layer was covered by a double side coated bleached Kraft-Glassine silicone paper liner. Patches were subsequently cut to the desired dimensions. Detailed composition of the fragrance-containing film of this example is given below:

| Materials (solids) | % (on a dry basis) |
|---|---|
| Polyurethane-1 | 69.74 |
| Fragrance | 30.22 |
| Erythrosine | 0.04 |
| Total | 100.00 |

1. A method for making an active layer to deliver a volatile substance, the method comprising:
providing a polymer solution including a polymer, a solvent, and the volatile substance;
applying the polymer solution to a side of a first release liner; and
drying the polymer solution to create the active layer.

2-3. (canceled)

4. A method for making a patch in accordance with claim 2.

5-7. (canceled)

8. A method for making a patch with a fractional adhesion layer to attach the patch to a surface in accordance with claim 4, the method further comprises applying a fractional adhesion layer to an opposite side of the barrier layer from the active layer, wherein the fractional adhesion layer has an adhesion area that is a fraction of a total area of a side of the barrier layer.

9-10. (canceled)

11. A method in accordance with claim 8, wherein applying the fractional adhesion layer includes:
applying an adhesive layer to an opposite side of the barrier layer from the active layer; and
applying an intermittent mask layer to cover a portion of the adhesive layer, the mask layer leaving adhesive exposed on the adhesion area of the barrier layer.

12-14. (canceled)

15. A method in accordance with claim 8, wherein the barrier layer is a double coated tape, each side of the double coated tape covered with an adhesive layer; and wherein positioning includes:
applying an intermittent mask layer to one adhesive layer of the double coated tape, the intermittent mask layer leaving adhesive exposed on the adhesion area of the barrier layer,
attaching the double coated tape to the active layer with the adhesive layer of the double coated tape opposite the one adhesive layer.

16-19. (canceled)

20. A method in accordance with claim 1, wherein the polymer is a polyurethane.

21. (canceled)

22. A method in accordance with claim 1, wherein the volatile substance includes at least one of an aromatherapy agent; an aromatherapy oil, a therapeutic agent, a deodorizer, a perfume, an insect repellant, a botanical extract, a botanical oil, and a masking odor.

23-31. (canceled)

32. A method for making a rate controlling active layer to deliver a volatile substance at a controlled rate in accordance with claim 1, wherein the polymer solution includes a rate controlling composition, and the active layer is a rate controlling active layer.

33-34. (canceled)

35. A method in accordance with claim 32, wherein the rate controlling composition includes at least one of wax, silica, kaolin, chalk, diatomaceous earth, bentonite, titanium dioxide, glass particulates, and metal particulates.

36-37. (canceled)

38. A device for delivering a volatile substance comprising:
an active layer, the active layer being a dried polymer solution having the volatile substance; and
a first release liner in direct contact with the active layer, and covering a side of the active layer.

39. (canceled)

40. A device in accordance with claim 38, wherein a polymer in the polymer solution is a polyurethane.

41. (canceled)

42. A device in accordance with claim 38, wherein the volatile substance includes at least one of an aromatherapy agent; an aromatherapy oil, a therapeutic agent, a deodorizer, a perfume, an insect repellant, a botanical extract, a botanical oil, and a masking odor.

43-50. (canceled)

51. A device in accordance with claim 38, wherein the active layer is a rate controlling active layer that includes a rate controlling composition that controls the rate of delivery of the volatile substance.

52-53. (canceled)

54. A device in accordance with claim 51, wherein the rate controlling composition includes at least one of wax, silica, kaolin, chalk, diatomaceous earth, bentonite, titanium dioxide, glass particulates, and metal particulates.

55. (canceled)

56. A patch for delivering a volatile substance in accordance with claim 38 further comprising:
a barrier layer disposed on an opposite side of the active layer from the first release liner.

57-60. (canceled)

61. A patch in accordance with claim 56 further comprising:
a first adhesive layer adjacent to an opposite side of the barrier layer from the active layer.

62-63. (canceled)

64. A patch in accordance with claim 61 further comprising:
an intermittent mask layer covering a portion of the first adhesive layer, the intermittent mask layer leaving adhesive exposed on a fraction of a total area of a side of the barrier layer.

65-66. (canceled)

67. A patch in accordance with claim 56 further comprising:
a second release liner attached to an opposite side of the barrier layer from the active layer.

68-69. (canceled)

70. A patch with multiple release liners for delivering a volatile substance at a controlled rate, the patch comprising:
an active layer having the volatile substance;
at least two release liner segments covering one side of the active layer; and
a barrier layer disposed on an opposite side of the active layer from the at least two release liner segments; wherein the release of the volatile substance depends upon the number of release liner segments removed from the active layer.

71. A patch in accordance with claim 70 further comprising:
a breathable layer disposed between the release liner segments and the active layer, the breathable layer hindering the rate of delivery of the volatile substance.

72-81. (canceled)
